(19) United States
(12) Patent Application Publication
(10) Pub. No.: US 2026/0213003 A1
(43) Pub. Date: Jul. 23, 2026

(54) SMART CRASH CART

(71) Applicant: Rohan BAJAJ, Brookline, MA (US)

(72) Inventor: Rohan BAJAJ, Brookline, MA (US)

(21) Appl. No.: 19/454,784

(22) Filed: Jan. 21, 2026

Related U.S. Application Data
(60) Provisional application No. 63/747,765, filed on Jan. 21, 2025.

(51) Int. Cl. G16H 40/20; G06K 7/10

(52) U.S. Cl. CPC G16H 40/20; G06K 7/10297

(57) ABSTRACT

Systems and methods of a smart crash cart are disclosed herein. The smart crash cart may include a frame. The smart crash cart may include a plurality of drawers within the frame. The smart crash cart may include one or more radio signal sensors within the drawers. The one or more radio signal sensors may be configured to sense radio signals from one or more chips disposed on one or more items in the drawers. Each chip may contain information relating to an item of the one or more items. The information may include an expiration date of the item. The smart crash cart may include a display disposed on the frame configured to display a listing of one or more items in the plurality of drawers. The smart crash cart may include a battery electrically connected to at least the one or more radio signal sensors and the display.

### CROSS-REFERENCE TO RELATED APPLICATIONS

This application claims priority to U.S. Provisional Application No. 63/747,765, filed on Jan. 21, 2025, which is incorporated by reference in its entirety.

### FIELD OF DISCLOSURE

This application generally relates to a smart crash cart configured to track items within the smart crash cart.

### BACKGROUND

Crash carts contain essential medical supplies and equipment used in emergency situations particularly for resuscitation and managing cardiac arrest. These carts are strategically designed to ensure quick access to life-saving tools and medications. After each use, the carts need to be inventoried to determine which tools and medications have been used and restocked accordingly.

### SUMMARY

In some embodiments, a smart crash cart is provided. The smart crash cart may include a frame. The smart crash cart may include a plurality of drawers within the frame. The smart crash cart may include one or more radio signal sensors within the plurality of drawers. The one or more radio signal sensors may be configured to sense one or more radio signals from one or more chips disposed on one or more items in the plurality of drawers. Each chip may contain information relating to an item of the one or more items. The information may include an expiration date of the item. The smart crash cart may include a display disposed on the frame configured to display a listing of one or more items in the plurality of drawers. The smart crash cart may include a battery electrically connected to at least the one or more radio signal sensors and the display.

In some embodiments, a method is provided. The method may include providing one or more chips on one or more items in a smart crash cart. Each of the one or more chips may contain information relating to an item of the one or more items. The information may include an expiration date of the item. The method may include sensing, via one or more radio signal sensors, radio signals from the one or more chips. The method may include determining, by a computing system including at least one processor, a difference between a current date and the expiration date of the item. The method may include comparing, by the computing system, the difference with a threshold difference. The method may include determining, by the computing system, the difference is at the threshold difference. The method may include notifying, via a push notification by the computing system, a central server of the expiration date of the item, the central server configured to communicate with the smart crash cart.

### BRIEF DESCRIPTION OF THE FIGURES

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1A depicts an illustrative smart crash cart, in accordance with example embodiments.

FIG. 1B depicts an illustrative smart crash cart with a touch screen mounted on a top of the smart crash cart, in accordance with example embodiments.

FIG. 2 depicts a view of an illustrative drawer of the smart crash cart, in accordance with example embodiments.

FIG. 3 depicts an illustrative radio signal sensor disposed on the smart crash cart, in accordance with example embodiments.

FIG. 4 depicts an illustrative smart crash cart including a waste bin disposed on a side of the smart crash cart, in accordance with example embodiments.

FIG. 5A depicts an illustrative computing environment, in accordance with example embodiments.

FIG. 5B depicts an example user interface including a plurality of smart crash carts in a network, in accordance with example embodiments.

FIG. 6 depicts an illustrative method for inventory tracking of a smart crash cart, in accordance with example embodiments.

FIG. 7 depicts an illustrative computing device, in accordance with example embodiments.

The features of the present disclosure will become more apparent from the detailed description set forth below when taken in conjunction with the drawings. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Disclosed herein is systems and methods of a smart crash cart. One or more techniques described herein improve upon conventional systems of crash carts by providing real-time inventory tracking of the smart crash cart. For example, a smart crash cart is disclosed which may include radio signal sensors configured to detect radio signal chips disposed on one or more items within the smart crash cart. A crash cart may also be known as a code cart, such that reference to a smart crash cart also refers to a smart code cart. The radio signal chips may include information relating to a corresponding item, such as an expiration date. The smart crash cart may track the expiration date of each item such that a real-time status of the one or more items can be determined. Instead of requiring manual inventorying, the smart crash cart may provide an accurate status of each item in the smart crash cart and may further provide information regarding a number of items remaining in the smart crash cart.

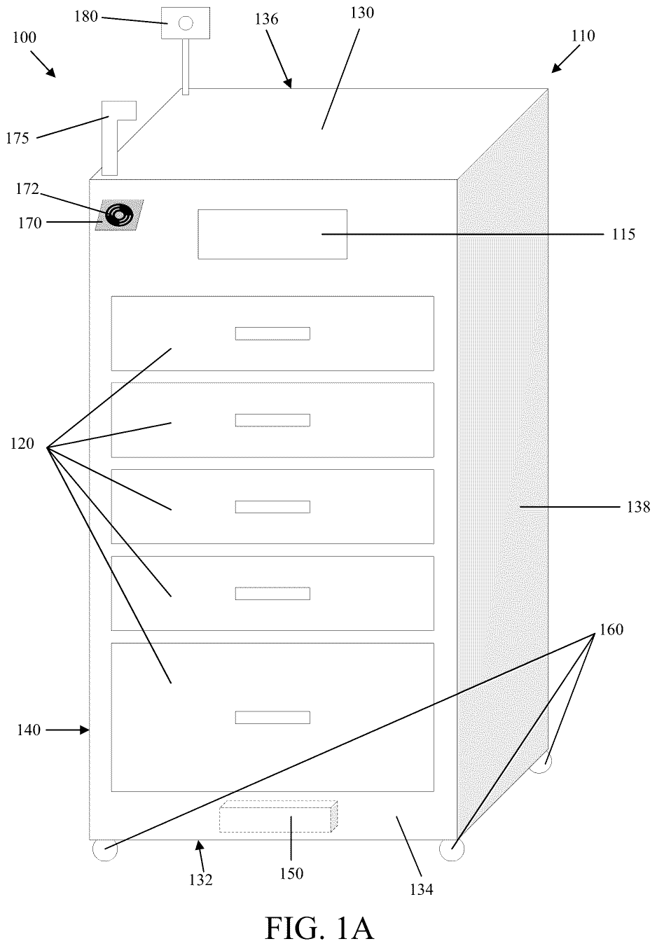

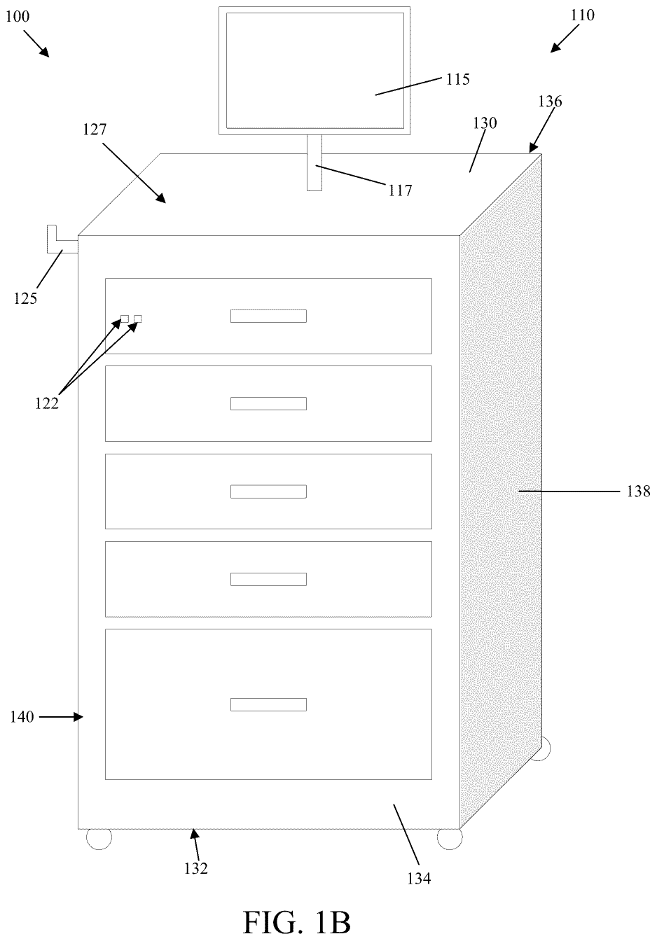

FIG. 1A is an illustrative smart crash cart 100, in accordance with example embodiments. The smart crash cart 100 may include a frame 110. The frame 110 may include a top side 130, a bottom side 132, a front side 134, a back side 136, a right side 138, and a left side 140. A plurality of drawers 120 may be disposed within the frame 110 on a side of the frame 110. In some embodiments, the plurality of drawers 120 may be disposed on the front side 134 of the frame 110. The plurality of drawers 120 is not limited by the number of drawers depicted in FIG. 1A and may comprise any number of drawers desired to contain the items in a crash cart.

The smart crash cart 100 may include a display 115 disposed on the frame 110. In some embodiments, the display 115 may be disposed on the front side 134 of the frame 110. The display 115 may be electrically connected to a computing system of smart crash cart 100. In some embodiments, display 115 may be disposed on the top side 130 of the smart crash cart 100, as shown in FIG. 1B. The display 115 may include a graphical user interface (GUI). In some embodiments, the display 115 may include a touch screen to provide manipulation of and selection on the GUI. The computing system may be configured to generate one or more alerts and/or notifications regarding one or more items in smart crash cart 100 and display the one or more alerts and/or notifications via display 115. The display 115 may use one or more icons to display the information generated by the computing system. The display 115 may be configured to display information regarding the smart crash cart 100, such as a listing of items in the smart crash cart 100. For example, when the computing system determines an item in the smart crash cart 100 is expired, display 115 may display a listing of the item designated as expired.

The display 115 may be mounted on a mount 117 disposed on the frame 110. The mount 117 may be a moveable mount. In some embodiments, the movable mount may be configured to allow repositioning of the display 115 to improve visibility and usability during emergency situations. The movable mount may preserve the original crash cart form factor while providing flexibility in display positioning. In some embodiments, the display 115 may be a high-resolution touchscreen with a brightness of 400 nits or greater to provide visibility in various lighting conditions encountered in hospital environments. The movable mount may allow the display 115 to be adjusted along one or more axes, enabling clinicians to position the display at an optimal viewing angle during a code. In some embodiments, the movable mount may include an articulating arm that permits rotation, tilting, and extension of the display 115 relative to the frame 110. The touchscreen may include a button or icon which when selected by a user is configured to initiate a notification to a central pharmacy.

The smart crash cart 100 may include one or more visual LED indicators 122 disposed on an exterior face of each of the plurality of drawers 120. In some embodiments, the LED indicators 122 may be configured to provide immediate visual identification of drawer status to clinicians and staff. A green LED indicator may signify that the corresponding drawer is fully stocked and in compliance, indicating that all items within the drawer are present and have not expired. A red LED indicator may signify that one or more items within the corresponding drawer are missing, expired, or require verification. The LED indicators 122 may be electrically connected to the computing system of smart crash cart 100, which may control the state of each LED indicator 122 based on information received from the one or more radio signal sensors within the plurality of drawers 120. In some embodiments, the computing system may update the LED indicator state in real-time as items are removed from or placed into the drawers. The drawer-level LED indicators 122 may streamline medication and item restocking by enabling staff to quickly identify which drawers require attention without needing to consult the display 115 or systematically opening individual drawers. In some embodiments, the LED indicators 122 may include additional colors or patterns to convey other status information, such as an amber indicator to signify that an item within the drawer is approaching its expiration date. The LED indicators 122 may be positioned at a location on the exterior face of each drawer that is visible when the smart crash cart 100 is approached from the front side 134 of the frame 110.

The smart crash cart 100 may include one or more built-in mounting points 125 configured to securely hold emergency equipment. In some embodiments, the mounting points 125 may include a dedicated mount for a backboard used for chest compressions during cardiopulmonary resuscitation (CPR). The backboard mount may be positioned on the frame 110 to allow rapid access and deployment during a code. The smart crash cart 100 may include a mount configured to secure an oxygen tank on a side of the frame 110, such as the right side 138 or the left side 140. In some embodiments, the oxygen tank mount may include straps, brackets, or clamps configured to retain the oxygen tank during transport of the smart crash cart 100. In some embodiments, the top side 130 of the frame 110 may include a flat surface 127 configured for placing equipment or medications during a code. The flat surface 127 may provide a workspace for clinicians to stage items removed from the plurality of drawers 120 or to prepare medications for administration. The built-in mounting points 125 may be integrally formed with the frame 110 or may be separately manufactured components affixed to the frame 110.

The smart crash cart 100 may include a battery 150. The battery 150 may provide backup power for the smart crash cart 100. The battery 150 may be a rechargeable battery electrically connected to the smart crash cart 100 such that battery 150 may charge when smart crash cart 100 is electrically connected to an external power source. The battery 150 may be connected to at least one of display 115, one or more radio signal sensors 320, one or more radio signal sensors 420, smart crash cart 100, and any desired electrical equipment on smart crash cart 100. Thus, operating power may be provided by battery 150 when smart crash cart 100 is not electrically connected to an external power source. One of ordinary skill in the art will understand that battery 150 may be disposed anywhere in smart crash cart 100.

The smart crash cart 100 may include a shelf 170. The shelf 170 may include a radio signal sensor 172. The shelf 170 may be retractable such that the shelf 170 may be withdrawn into the smart crash cart 100. The radio signal sensor 172 in the shelf 170 may be configured to sense a radio signal chip adjacent to the shelf 170. The radio signal sensor 172 may receive a radio signal transmitted from the radio signal chip. The shelf 170 may be used to register the radio signal chip on an item prior to the item being placed in the smart crash cart 100.

The smart crash cart 100 may include a vision-based scanner 175. The vision-based scanner 175 may be a QR code scanner or a barcode scanner. The vision-based scanner 175 may include a camera configured to take an image of a code, such as a QR code or barcode. The computing system of the smart crash cart 100 may determine information based on the scanning of the code. For example, an item having a code may be scanned such that the smart crash cart 100 may determine that the item is dobutamine. The vision-based scanner 175 may be used as part of the registration process of the items. For example, when an item is provided to be placed in the smart crash cart 100, a radio signal chip may be disposed on the item. The item may be placed adjacent to the shelf 170 which registers the radio signal chip. A code on the item may then be scanned by the vision-based scanner 175. The computing system may correlate the radio signal chip with the information determined about the item via the vision-based scanner 175. The item may be placed in the smart crash cart 100. Subsequently, when the radio signal chip is detected by a radio signal sensor, the smart crash cart 100 may determine the information relating to the radio signal chip. The vision-based scanner 175 may be retractable such that the vision-based scanner 175 may be withdrawn into the smart crash cart 100.

The smart crash cart 100 may include a camera 180. The camera 180 may be a computer vision camera or a camera included on a handheld device, e.g., a phone or tablet. The camera 180 may be retractable such that the camera 180 may be withdrawn into the smart crash cart 100. In some embodiments, each of the plurality of drawers 120 may include a camera 180 to provide imaging of each drawer 120. The camera 180 may be configured to scan at least a label of an item to determine information relating to the item. The camera 180 may provide an image to the computing system of smart crash cart 100. The computing system may extract text from the image in order to determine what the item is. In some embodiments, the image may be provided to an application on a handheld device to extract text from the image prior to transmitting the information to the computing system. The handheld device may be connected to the computing system of the smart crash cart 100 via at least one of WiFi, cellular, and/or Bluetooth™. The smart crash cart 100 may include a wireless transceiver in communication with the computing system of smart crash cart 100 in order to receive information from and/or transmit information to one or more external computing devices (e.g., the handheld device). In some embodiments, the computing system of smart crash cart 100 may be configured to recognize a medication via the camera 180 using direct visual recognition. Thus, when a radio signal chip is disposed on an item and registered via shelf 170, the camera 180 may provide the desired information of the item to allow the computing system to correlate the information with the radio signal chip disposed on the item. In some embodiments, the computing system may utilize a machine learning model trained to recognize medications and reconcile the medication labels and/or expiration information. The machine learning model may be configured to prompt restocking of smart crash cart 100 and/or reordering of medications running low in smart crash cart 100.

In some embodiments, during the intake process, a user may apply a radio signal chip to an item, scan the medication label using the camera 180, and tap the item on a radio signal reader to register the item in the smart crash cart 100. The computing system may extract medication information from the captured image, including medication name, expiration date, lot number, and manufacturer information. In some embodiments, the computer vision camera may utilize optical character recognition (OCR) to extract text from medication labels. The computing system may correlate the extracted information with the registered radio signal chip, thereby associating the chip with the medication metadata. This intake workflow may reduce manual data entry that would otherwise be performed by pharmacists or technicians, and may improve accuracy by automating the capture of medication information directly from the label.

The information determined by the vision-based scanner 175 and/or camera 180 may be displayed via display 115. The display 115 may include an icon representing an option to manually override the scanned data. For example, if a user registers a radio signal chip and enters the information relating to the item via vision-based scanner 175 and/or camera 180, the user may still be able to confirm that the scanned information is correct and correct it if it is not. Thus, entering the items into the smart crash cart 100 may be streamlined and allow for shorter time spent stocking the smart crash cart 100.

The bottom side 132 of the frame 110 may include a plurality of wheels 160 to provide mobility to the smart crash cart 100. The plurality of wheels 160 may be any type of suitable wheels, such as caster wheels. The wheels 160 may include a locking mechanism to lock the wheels in place when desired, such as during storage and/or while being used for a patient. The locking mechanism may be foot-activated.

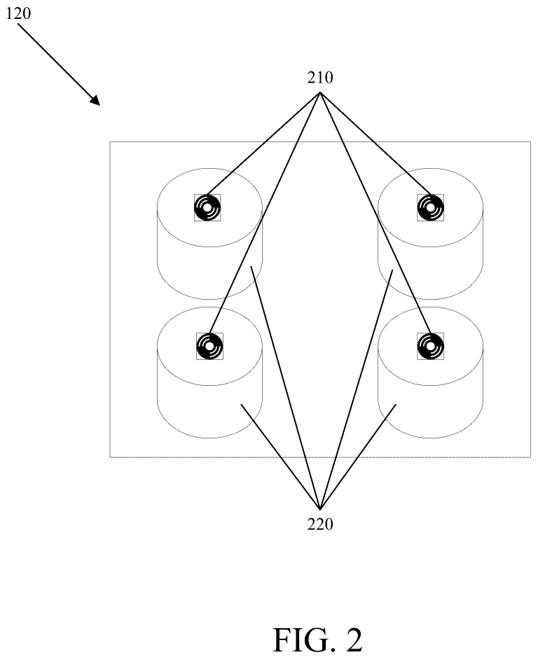

FIG. 2 is a view of an illustrative drawer 120 containing one or more items 220 with one or more chips 210 disposed on the one or more items 220. The one or more items 220 may be any items found in or on a crash cart and can include medications, medical instruments including advanced cardiovascular life support (ACLS) instruments, airway equipment, compression back board, defibrillator, and defibrillator pads. The chips 210 may be radio signal chips. The chips 210 may be at least one of radio frequency identification (RFID) chips, near-field communication (NFC) chips, and Bluetooth™ chips. The chips 210 may be up to about 10 cm² in size. In some embodiments, the chips 210 may be about 2 cm² in size. Each of the one or more items 220 may have a chip 210 disposed on it with information relating to the item. For example, an item of dobutamine may have a chip 210 disposed on it with information loaded onto the chip 210 indicating that the item is dobutamine along with its expiration date. The information loaded onto the chip 210 may also include one or more of a national drug code, a lot number of the item, an indication of the manufacturer and/or the distributor, medication concentration, or critical administration instructions. Smart crash cart 100 may use the information to identify issues with the medication. For example, if a medication is recalled based on the lot number of the medication, smart crash cart 100 may identify which medications in smart crash cart 100 need to be replaced. In some embodiments, central server 520 may be configured to determine which medications across the whole system of smart crash carts 100 need to be replaced. As central server 520 may be configured to track the location of each smart crash cart 100, central server 520 may be able to identify the locations of each medication that needs to be replaced.

In some embodiments, the smart crash cart 100 may include standardized plastic trays disposed within the plurality of drawers 120. In some embodiments, each drawer 120 may include a plastic insert configured to accommodate a hospital's preferred medication layout. The plastic trays may include a plurality of compartments or dividers arranged to hold specific medications or items in predetermined positions within the drawer 120. The standardized layout may allow clinicians to locate medications more quickly during emergency situations by providing consistent placement of items across multiple smart crash carts 100 within a healthcare facility. In some embodiments, the plastic trays may be removable from the drawers 120 to facilitate cleaning, replacement, or reconfiguration. The compartments within the plastic trays may be sized and shaped to accommodate various medication containers, vials, syringes, or other items typically stored in a crash cart. In some aspects, the plastic trays may include labeling areas or indicia corresponding to each compartment to identify the intended contents. The standardized tray configuration may support training workflows by enabling staff to become familiar with item locations that remain consistent across the fleet of smart crash carts 100. In some embodiments, different tray configurations may be provided for adult, pediatric, or neonatal crash carts, with compartment arrangements tailored to the specific clinical needs of each patient population.

Radio signal chips disposed on the one or more items may provide for accurate inventorying of the smart crash cart. A radio signal chip may transmit an electromagnetic signal carrying the information relating to that radio signal chip. The radio signal sensor corresponding to the drawer holding the radio signal chip may detect the electromagnetic signal and convert it to an electrical signal. The computing system may then determine based on the electrical signal the information relating to the radio signal chip. The radio signal sensor for each drawer may be configured to detect only the radio signal chips within its corresponding drawer. This allows the smart crash cart to determine which drawer each item is in based on which radio signal sensor sensed which radio signal chip. Thus, the smart crash cart is enabled to accurately determine which items are in each drawer and how many items are in each drawer.

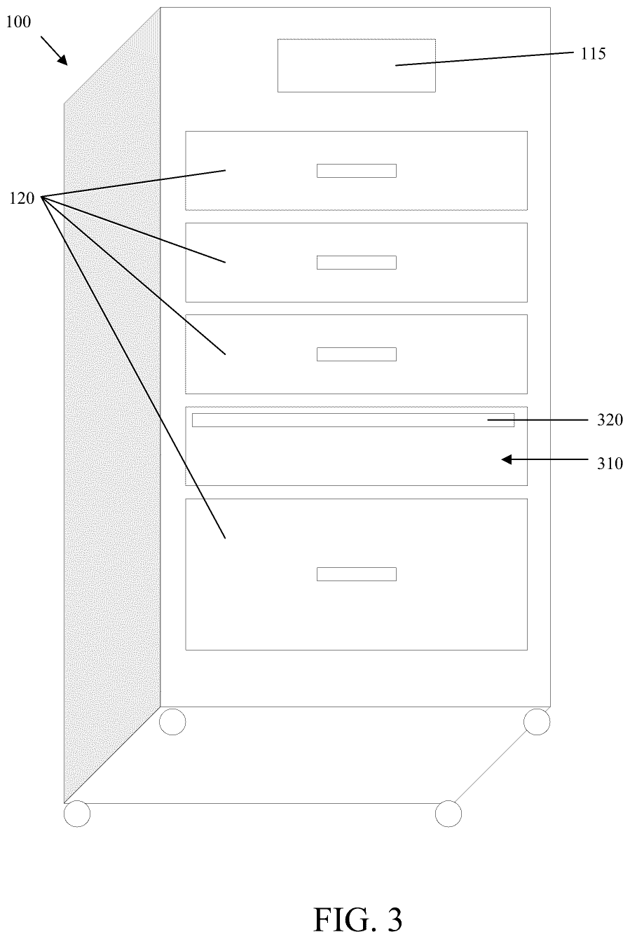

FIG. 3 is a view of an illustrative smart crash cart 100 depicting a radio signal sensor 320 disposed within drawer area 310. The radio signal sensor 320 may be disposed anywhere within the drawer area 310. In some embodiments, as shown, the radio signal sensor 320 may be disposed on an upper surface defining an upper bound of drawer area 310. Each of the plurality of drawers may have a corresponding radio signal sensor 320. In some embodiments, the radio signal sensor 320 may be located within one of the plurality of drawers 120. The radio signal sensor 320 may be configured to detect all the radio signal chips within its corresponding drawer of the plurality of drawers 120. The radio signal sensor 320 may detect electromagnetic signals transmitted from one or more radio signal chips. The radio signal sensor 320 may be directed such that it may determine only the radio signal chips within its corresponding drawer area 310. The radio signal sensor may have a read range of between about 0.5 inches to about 117 inches. In some embodiments, the radio signal sensor 320 may have a read range of between about 2 inches and about 12 inches. If an item with a radio signal chip is removed from the corresponding drawer area 310, the radio signal sensor 320 may no longer detect the electromagnetic signal transmitted from the radio signal chip. Thus, when an item is placed in a drawer, a radio signal sensor corresponding to the drawer can detect the presence of a chip on the item and update the smart crash cart 100 system to include the detected item along with the information related to the detected item. Additionally, the radio signal sensor 320 may detect when an item is removed from the corresponding drawer. While other sensors may be used, radio signal sensors combined with radio signal chips provides for specific identification of a moved item without the need for a map of where items are, as may be required for infrared sensors, for example, which sense the location of objects based on thermal imaging.

The smart crash cart 100 may include drawer-level spatial sensing configured to determine the location of items within individual drawers. In some embodiments, the computing system may calculate a distance from a radio signal sensor to an item based on signal strength received from the radio signal chip disposed on the item. The drawer-level sensing information may be visualized on the display 115, showing which item is located in which drawer. In some embodiments, the central server 520 may also display the drawer-level location information, supporting medication tracking, staff training, and replenishment workflows. The radio signal sensors may include radio signal sensors 320 configured in various arrangements to provide spatial sensing within the drawers. In some embodiments, radio signal sensors may be mounted at the top and bottom of the smart crash cart 100, with hollowed metal drawers acting as an RF shield between drawer areas. In some embodiments, an radio signal sensor may be disposed in each drawer, with items placed directly on or adjacent to the radio signal sensor and metal faces on sides of the drawer. In some embodiments, a single radio signals sensor may be mounted at the top or bottom of the smart crash cart 100 with a multi-foot read range configured to sense items across multiple drawers 120.

The smart crash cart 100 may include a metal exterior configured to provide radio frequency isolation. The metal exterior may form a Faraday-cage-like environment that blocks external radio signals from interfering with the radio signal sensors within the smart crash cart 100. This RF isolation may reduce or prevent false detections that could otherwise occur from radio signal chips on items in neighboring crash carts or other nearby equipment. The metal enclosure may be configured to block external RFID signals from outside the smart crash cart 100 while preserving internal sensing capabilities of the radio signal sensors within the drawers. In some embodiments, the plurality of drawers 120 may include metal faces or metal components configured to provide RF shielding between adjacent drawers, allowing each radio signal sensor 320 to detect only the radio signal chips within its corresponding drawer area 310 without interference from chips in other drawers.

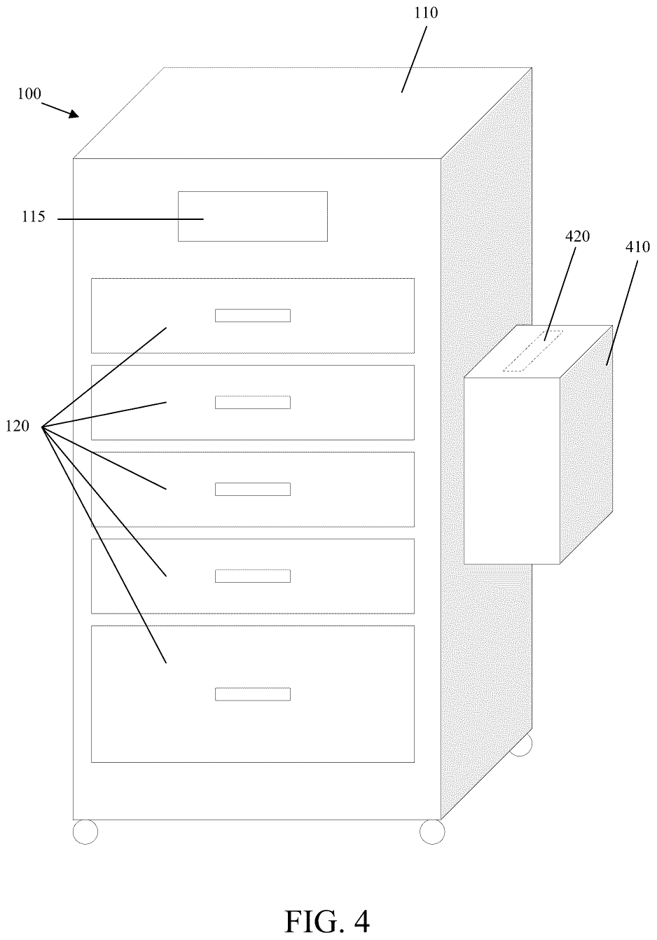

FIG. 4 is a view of an illustrative smart crash cart 100 including a waste bin 410 disposed on a side of the smart crash cart 100. In some embodiments, the smart crash cart 100 may have a waste bin 410 disposed on the frame 110 of the smart crash cart 100. The waste bin 410 may provide a place for disposing of used items taken from the plurality of drawers 120. The waste bin 410 may include one or more radio signal sensors 420. The one or more radio signal sensors 420 may be substantially similar to the radio signal sensors 320 provided in FIG. 3. The one or more radio signal sensors 420 may be disposed on any surface of the waste bin 410. In some embodiments, the one or more radio signal sensors 420 may be disposed on a lid of waste bin 410. The one or more radio signal sensors 420 may detect the presence of a chip disposed on an item when the item is placed in the waste bin 410. The computing system of smart crash cart 100 may be configured to match the item detected in the waste bin 410 with an item removed from one of the plurality of drawers 120. Thus, the smart crash cart 100 can account for an item removed from the plurality of drawers 120. In some embodiments, the smart crash cart 100 may generate a log of items based on which items are detected by the one or more radio signal sensors 420 of the waste bin 410. The log may be displayed on the display 115. The log may provide an improved system for restocking a crash cart and for billing the appropriate patient for items used.

The waste bin 410 may serve as a confirmation mechanism for medication administration during emergency situations. During a code, medications may be removed from the plurality of drawers 120 and staged at a bedside without being immediately administered to a patient. In such cases, detection of item removal from a drawer alone may not reliably indicate that the medication was definitively administered. The one or more radio signal sensors 420 disposed in the waste bin 410 may provide confirmation that an item has been used by detecting when the item is disposed of after administration. The smart crash cart 100 may distinguish between items that have been removed from the drawers and items that have been confirmed as administered based on detection in the waste bin 410. In some embodiments, the waste bin 410 may be integrated with the operating system of the smart crash cart 100 such that the smart crash cart 100 may correlate waste bin detections with drawer removal events to provide a more accurate record of medication usage.

The waste bin 410 may be configured as an attachment disposed on a side of the frame 110 of the smart crash cart 100. In some embodiments, the waste bin 410 may be removable from the frame 110 for cleaning or replacement. The one or more radio signal sensors 420 may be positioned to detect radio signal chips on items as the items pass through an opening of the waste bin 410 or when the items are present within the waste bin 410. The computing system may update the log of removed items to indicate which items have been placed in the waste bin 410, thereby providing a record of administered medications. In some embodiments, the log may differentiate between items that have been removed from drawers but not yet placed in the waste bin 410 and items that have been confirmed as disposed. This differentiation may support quality control workflows by enabling staff to verify that removed medications were actually administered rather than misplaced or returned to an incorrect location.

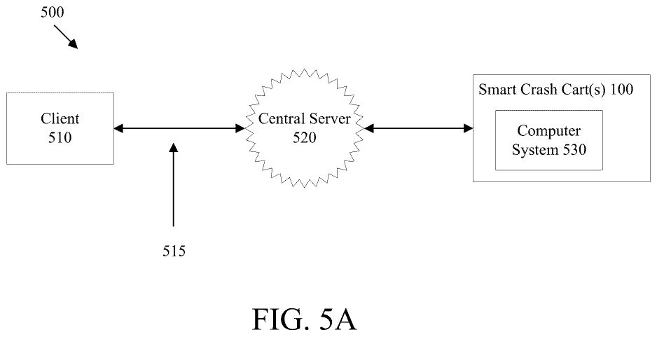

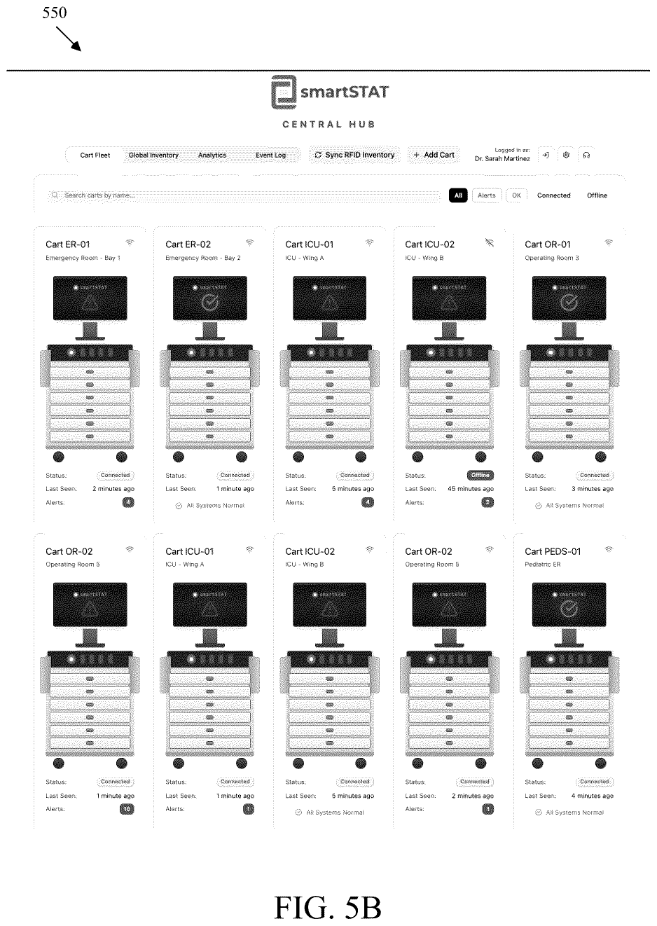

FIG. 5A is a block diagram illustrating an illustrative computing environment 500 for a smart crash cart 100, according to example embodiments. As shown, the computing environment 500 may include a client 510, a central server 520, and one or more smart crash carts 100 communicating via network 515.

Network 515 may be of any suitable type, including individual connections via the Internet, such as cellular or Wi-Fi networks. For example, the cellular connection may be via 5G or LTE telecommunications chips. In some embodiments, network 515 may connect terminals, services, and mobile devices using direct connections, such as radio frequency identification (RFID), near-field communication (NFC), Bluetooth™, low-energy Bluetooth™ (BLE), Wi-Fi™, ZigBee™, cellular (e.g., LTE and/or 5G), ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

Network 515 may include any type of computer networking arrangement used to exchange data. For example, network 515 may be the Internet, a private data network, virtual private network using a public network and/or other suitable connection(s) that enables components in computing environment 500 to send and receive information between the components of computing environment 500.

Clients 510 may be representative of one or more computing devices. For example, clients 510 may be representative of a desktop terminal, a laptop computer, a tablet computer, a smartphone, etc. Any type of computing device that allows an access to the central server 520 through the network 515 should be considered within the scope of this disclosure. Furthermore, the functionality described within this disclosure can be distributed in any fashion, i.e., functionality of the central server 520 may be performed by one or more clients 510 and vice versa.

As described above and in further details below, the smart crash cart 100 may include a computer system 530. Computer system 530 may include multiple software modules configured to track the date for determining if an expiration date is approaching, log items placed into and removed from the smart crash cart 100, track the location of the smart crash cart 100, and input information relating to an item into the computing system of smart crash cart 100. The one or more software modules are collections of code or instructions stored on a media (e.g., memory of smart crash cart 100) that represent a series of machine instructions (e.g., program code) that implements one or more algorithmic steps. The machine instructions may be the actual computer code the processor of smart crash cart 100 interprets to implement the instructions or, alternatively, may be a higher level of coding of the instructions that are interpreted to obtain the actual computer code. The one or more software modules may also include one or more hardware components. One or more aspects of an example algorithm may be performed by the hardware components (e.g., circuitry) itself, rather than as a result of the instructions. Functionality of each module may be discussed in further detail herein, in conjunction with FIGS. 1-4 and 6.

In some embodiments, smart crash cart 100 may utilize one or more machine learning models to perform the functions of the computer system 530. The one or more machine learning models may be trained to perform one or more of the following functions: recognize medications using one or more of RFID, NFC, or computer vision; determine an expiration date for each item placed in smart crash cart 100; log items removed from smart crash cart 100; track an inventory status of items in smart crash cart 100; predict for each category of items when the item should be restocked (e.g., based on expiration date and/or rate of use of a category); notify client 510 of a need for restocking an item in smart crash cart 100; or communicate with one or more other smart crash carts within a network. The smart crash cart 100 may be configured to perform hybrid sensing using two or more of RFID, NFC, or computer vision to provide redundancy and accuracy even if one of the sensing components fails.

FIG. 5B depicts an example user interface 550 including a plurality of smart crash carts 100 in a network, in accordance with example embodiments. As shown, computer system 530 may display via user interface 550 that Cart ER-01 has four alerts along with an indication of which drawers need attention while Cart ER-02 has an indication of “All systems normal.” The indication of which drawers need attention may include highlighting one or more drawers in red, for example. User interface 550 may be available to one or more of client 510, central server 520, or computer system 530. Accordingly, the status of each smart crash cart 100 in a network may be viewed by one or more users within the network.

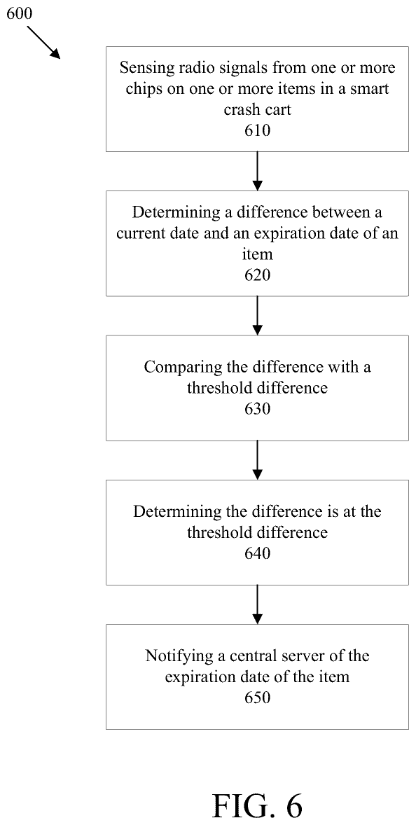

FIG. 6 is a flowchart illustrating method 600 for inventory tracking of a smart crash cart 100, in accordance with example embodiments. Method 600 may begin at step 610. One or more chips on one or more items in a smart crash cart may be provided. Each of the one or more chips may contain information relating to an item of the one or more items. The information may include an expiration date of the item.

At step 610, one or more radio signal sensors may sense radio signals from the one or more chips. The radio signal sensors may be at least one of RFID sensors, NFC sensors, and Bluetooth™ devices. The one or more radio signal sensors may each correspond to a drawer of the smart crash cart 100.

At step 620, the computing system of smart crash cart 100 may determine a difference between a current date and the expiration date of the item. Smart crash cart 100 may include a date tracking system to track the current date. With each item put into the smart crash cart 100, the smart crash cart 100 may log the information from the chip corresponding to the item. Smart crash cart 100 may track the difference between the current date and the detected expiration date of each item in the smart crash cart 100.

At step 630, the computing system of smart crash cart 100 may compare the difference between the current date and the expiration date of the item with a threshold difference. One of ordinary skill in the art will understand that the threshold difference may be a predetermined difference of any amount. In some embodiments, the threshold difference may be zero.

At step 640, smart crash cart 100 may determine, via the computing system, the difference is at the threshold difference. For example, if an expiration date for an item is Jan. 15, 2025, and the threshold difference is seven days, the computing system may determine the difference between a current date and the expiration date is at the threshold difference when the current date is Jan. 8, 2025.

At step 650, smart crash cart 100 may notify, via a push notification, a central server 520 of the expiration date of the item. The central server 520 may be configured to communicate with the smart crash cart 100 via network 515. Central server 520 may communicate the expiration date notification to one or more clients 510 in order to facilitate replacement of the item. In some embodiments, smart crash cart 100 may also provide an alert via the display on smart crash cart 100 of items approaching expiration. This may provide potential users vital information prior to using a specific crash cart. For example, if an expiration date is approaching for a specific medication, a user may be able to see via the display that the specific medication is approaching expiration. If that medication is important to a code for which the smart crash cart 100 is needed, the user may choose a different smart crash cart 100 if available.

Smart crash cart 100 may provide notifications to the central server 520 regarding the number of items in the smart crash cart 100. In some embodiments, central server 520 may communicate information received from smart crash cart 100 to one or more clients 510 via network 515. For example, during certain codes, certain medications must be administered regularly. In some instances, the code may extend for about 30 minutes or more. Depending on the length of the code, a necessary medication or category of items may be depleted, and a pharmacist may ordinarily need to be present to issue more of the medication or items. With the smart crash cart 100, as the medication is being depleted, smart crash cart 100 may notify central server 520 that the medication is being depleted. Central server 520 may then notify a client 510 corresponding to the pharmacist of the need of the medication and of the location of the smart crash cart 100. The pharmacist may then send more medication or more of the items to the location of the smart crash cart 100 in need of the medication.

The smart crash cart 100 may include a real-time central pharmacy notification system configured to alert pharmacy personnel when the smart crash cart 100 is in use during an emergency. In some embodiments, when a code occurs and the smart crash cart 100 is opened, the computer system 530 may automatically transmit a notification to the central server 520, which may in turn communicate with a central pharmacy workstation. The central pharmacy workstation may receive the notification and generate an audio-visual alert to notify pharmacy staff that a code is in progress and that the smart crash cart 100 may require attention. The audio-visual alert may include one or more of an audible tone, a visual indicator such as a flashing light or on-screen notification, or a combination thereof. In some embodiments, the notification may include information regarding the location of the smart crash cart 100 within the hospital or healthcare facility, enabling pharmacy staff to prepare medications or supplies that may be needed during the code. The display 115 of the smart crash cart 100 may include a button or icon configured to allow a user to actively notify the central pharmacy when the smart crash cart 100 requires attention during an emergency. For example, if a clinician determines that additional medications are needed during a code, the clinician may press the button on the touchscreen to transmit a request to the central pharmacy. The central server 520 may receive the request and communicate it to the central pharmacy workstation, where pharmacy staff may respond by dispatching the requested medications to the location of the smart crash cart 100. In some embodiments, the notification system may provide bidirectional communication, allowing pharmacy staff to acknowledge receipt of the notification or to communicate with clinicians at the smart crash cart 100 regarding medication availability or estimated delivery times.

The smart crash cart 100 may be configured to maintain a detailed, time-stamped record of item usage and removal events. In some embodiments, when an item is removed from one of the plurality of drawers 120, the computer system 530 may log the removal event along with a timestamp indicating the date and time at which the removal occurred. The time-stamped record may include information identifying the specific item removed, the drawer from which the item was removed, and the duration for which the item was outside of the smart crash cart 100. In some aspects, the computing system may also record when items are returned to the smart crash cart 100 or placed in the waste bin 410, with corresponding timestamps for each event. The time-stamped usage data may be transmitted to the central server 520 via network 515, where it may be aggregated and stored for subsequent analysis. In some embodiments, the central server 520 may generate reports based on the time-stamped records, providing visibility into medication usage patterns during emergency events. The time-stamped tracking may support post-event review, quality assurance workflows, and documentation of medication administration timelines during codes. Computer system 530 may be configured to only allow authorized users to restock items in the smart crash cart 100. For example, a user may be required to scan a badge prior to computer system 530 allowing the user to open a drawer of the smart crash cart. Computer system 530 may log the user that placed the item into the smart crash cart 100.

The computing system of smart crash cart 100 may communicate a status and location of the smart crash cart 100 with central server 520. For example, smart crash cart 100 may provide tracking information for each of the items within the smart crash cart 100 to the central server 520 via the one or more chips. The computing system of smart crash cart 100 may determine the location of the smart crash cart 100 via one or more of GPS location information, indoor positioning information, received signal strength indicator (RSSI) information, WiFi, Bluetooth™, and active and/or passive RFID, or equivalents thereof. Further, the one or more radio signal sensors in the smart crash cart 100 may enable the smart crash cart 100 to keep a log of which drawer and/or waste bin each item is in and provide that information to the central server 520. Such tracking of the items may provide improved inventorying and restocking. For example, after a smart crash cart 100 is used, the log of the different items used and items remaining in the drawers may be communicated to the central server 520. The central server 520 may communicate that information to a client 510 corresponding to a restocking department. Thus, the information necessary for restocking the smart crash cart 100 may be provided before the smart crash cart 100 is available for restocking providing for more efficient restocking of smart crash cart 100.

In some embodiments, the central server 520 may aggregate medication and supply usage data, along with expiration data, across a fleet of smart crash carts 100 within a hospital or healthcare facility. The computing system may apply one or more machine learning models to predict future depletion on a per-medication basis across all carts in the network. The machine learning models may analyze usage trends, upcoming expiration dates, and historical consumption patterns to generate reorder recommendations. In some embodiments, the reorder recommendations may account for a configurable buffer that hospitals can set based on their operational requirements. For example, the buffer may be two months prior to expiration to ensure that replacements are available in advance of an expiration date. The central server 520 may communicate the reorder recommendations to one or more clients 510, such as a pharmacy workstation or supply chain management system, to facilitate timely procurement of medications and supplies before stock levels become insufficient. In some embodiments, the machine learning models may identify patterns in medication usage that correlate with seasonal variations, patient population changes, or other factors affecting consumption rates. The system may continuously refine its predictions as additional usage data is collected from the fleet of smart crash carts 100, thereby improving the accuracy of reorder recommendations over time.

The computing system of smart crash cart 100 may detect via one or more radio signal sensors that an item was removed and later placed back into the smart crash cart 100. For example, a radio signal sensor in one drawer may detect removal of an item from the drawer and later detect replacement of the item into the drawer. The computing system may indicate in the log that an item was removed from the drawer and later replaced into the same drawer. As a further example, a second radio signal sensor in a second drawer may detect removal of an item from the second drawer. A third radio signal sensor in a third drawer may detect placement into the third drawer of the item removed from the second drawer. The computing system may indicate in the log that the item was removed from the second drawer and later placed in the third drawer. In some embodiments, the computing system may generate an alert regarding the replaced item and display it on the display of the smart crash cart 100 and/or communicate the alert to the central server 520.

The computing system of smart crash cart 100 may update the log based on which items are removed from the cart. The computing system may detect via one or more radio signal sensors that a first item was removed from the smart crash cart 100 and may update the log to include the first item. The log may be displayed via display 115. The computing system may detect that a second item with the same name as the first item was removed and may update the log to include a number corresponding to the first item. For example, if an item of dobutamine has been removed from the smart crash cart 100, the log may be updated to include one dobutamine being removed from the smart crash cart 100. If a second item of dobutamine is removed from the smart crash cart 100, the log may be updated to provide that two dobutamines have been removed from the smart crash cart 100. The updated log may be displayed on the display 115. Alternatively, the log may provide a listing of the separate items as being removed from the smart crash cart 100. In some embodiments, the computing system may indicate a remaining number of items of the item included in the log. For example, if the computing system has determined that there are six items of dobutamine in the smart crash cart 100 via the radio signal sensors, when two are removed, the computing system may indicate that there are four remaining items of dobutamine.

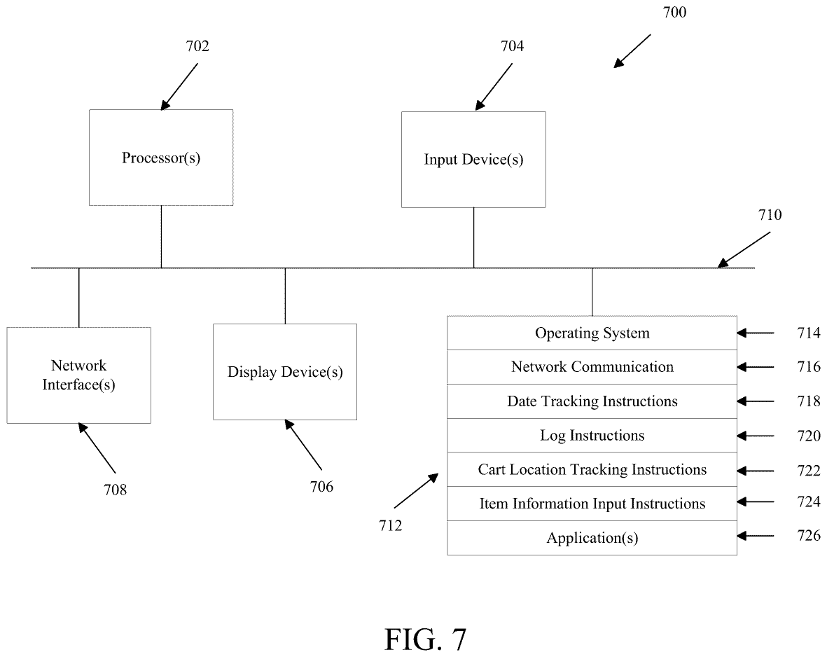

FIG. 7 shows a block diagram of an example computing device 700 that implements various features and processes, according to example embodiments of this disclosure. For example, computing device 700 may function as the central server 520, the clients 510, the smart crash cart 100, or a portion or combination thereof in some embodiments. The computing device 700 may also perform one or more steps of the method 600. The computing device 700 may be implemented on any electronic device that runs software applications derived from compiled instructions, including without limitation personal computers, servers, smart phones, media players, electronic tablets, game consoles, email devices, etc. In some implementations, the computing device 700 includes one or more processors 702, one or more input devices 704, one or more display devices 706, one or more network interfaces 708, and one or more computer-readable media 712. Each of these components may be coupled by a bus 710.

Display device 706 includes any display technology, including but not limited to display devices using Liquid Crystal Display (LCD) or Light Emitting Diode (LED) technology. Display device 706 may be display 115. Processor(s) 702 uses any processor technology, including but not limited to graphics processors and multi-core processors. Input device 704 includes any known input device technology, including but not limited to a keyboard (including a virtual keyboard), mouse, track ball, and touch-sensitive pad or display. Bus 710 includes any internal or external bus technology, including but not limited to ISA, EISA, PCI, PCI Express, USB, Serial ATA or FireWire. Computer-readable medium 712 includes any non-transitory computer readable medium that provides instructions to processor(s) 702 for execution, including without limitation, non-volatile storage media (e.g., optical disks, magnetic disks, flash drives, etc.), or volatile media (e.g., SDRAM, ROM, etc.).

Computer-readable medium 712 includes various instructions 714 for implementing an operating system (e.g., Mac OS®, Windows®, Linux). The operating system may be multi-user, multiprocessing, multitasking, multithreading, real-time, and the like. The operating system performs basic tasks, including but not limited to: recognizing input from input device 704; sending output to display device 706; keeping track of files and directories on computer-readable medium 712; controlling peripheral devices (e.g., disk drives, printers, etc.) which can be controlled directly or through an I/O controller; and managing traffic on bus 710. Network communications instructions 716 establish and maintain network connections (e.g., software for implementing communication protocols, such as TCP/IP, HTTP, Ethernet, telephony, etc.).

Date tracking instructions 718 includes instructions that implement the disclosed process for tracking the date in relation to an expiration date of an item, as described throughout this disclosure. Log instructions 720 includes instructions that implement the disclosed process for logging items in the smart crash cart 100 along with logging removal and placement of the items within one or more of the drawers and/or the waste bin. Cart location tracking instructions 722 includes instructions that implement the disclosed process for tracking the location of each smart crash cart, as described throughout this disclosure. Item information input instructions 724 includes instructions that implement the disclosed process for receiving information relating to an item and correlating the information with a registered radio signal chip. Application(s) 726 may comprise an application that uses or implements the processes described herein and/or other processes. The processes may also be implemented in the operating system.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment. In one embodiment, this may include Python. The computer programs therefore are polyglots.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as a CRT (cathode ray tube), LCD (liquid crystal display), OLED (organic light emitting diode), or LED (light emitting diode) monitor for displaying information to the user and a keyboard and a pointing device such as a mouse, a trackball, or a touchscreen by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

As used herein, the term “about” means plus or minus 10% of the numerical value of the number with which it is being used. Therefore, about 10 cm² means in the range of 9 cm² to 11 cm².

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A smart crash cart system comprising:
a frame;
a plurality of drawers within the frame;
one or more first radio signal sensors within the plurality of drawers, the one or more first radio signal sensors configured to sense one or more radio signals from one or more chips disposed on one or more items in the plurality of drawers, wherein each chip contains information relating to an item of the one or more items, the information including an expiration date of the item;
a display disposed on the frame configured to display a listing of one or more items in the plurality of drawers;
a waste bin disposed on the frame, the waste bin comprising one or more second radio signal sensors configured to sense one or more radio signals from one or more chips disposed on one or more items in the waste bin; and
a battery electrically connected to at least the one or more first radio signal sensors, the one or more second radio signal sensors, and the display.

2. The smart crash cart system of claim 1, the one or more first radio signal sensors comprising at least one of radio frequency identification (RFID) sensors, near-field communication (NFC) sensors, and Bluetooth sensors.

3. The smart crash cart system of claim 1, further comprising:
a computing system comprising at least one processor, the computing system configured to communicate with a central server, the central server configured to track the one or more items in the plurality of drawers via the one or more chips, the computing system electrically connected to the display, the computing system electrically connected to the battery.

4. The smart crash cart system of claim 3, wherein the computing system is configured to:
track the expiration date of each of the one or more items, and
issue a push notification to the central server, the push notification comprising a notification of an approaching expiration date of one of the one or more items.

5. The smart crash cart system of claim 3, further comprising:
a camera disposed on the frame, the camera configured to scan at least a label of an item of the one or more items, wherein the computing system is configured to extract text from the label captured by the camera to determine the item.

6. The smart crash cart system of claim 1, wherein the one or more chips are no more than about 10 cm².

7. The smart crash cart system of claim 1, wherein the one or more items comprise at least one of medications, airway equipment, and defibrillator equipment.

8. A computer-implemented method comprising:
providing one or more chips on one or more items in a smart crash cart, each of the one or more chips containing information relating to an item of the one or more items, the information including an expiration date of the item;
sensing, via one or more radio signal sensors, radio signals from the one or more chips;
determining, by a computing system comprising at least one processor, a difference between a current date and the expiration date of the item;
comparing, by the computing system, the difference with a threshold difference;
determining, by the computing system, the difference is at the threshold difference;
notifying, via a push notification by the computing system, a central server of the expiration date of the item, the central server configured to communicate with the smart crash cart;
sensing, via the one or more radio signal sensors, removal of the item from the smart crash cart;
sensing, via the one or more radio signal sensors, replacing of the item in the smart crash cart;
generating, by the computing system, an alert of the replaced item; and
transmitting, by the computing system, the alert to the central server.

9. The computer-implemented method of claim 8, further comprising:
tracking a location of the one or more items via the one or more chips.

10. The computer-implemented method of claim 8, further comprising:
generating, by the computing system, a log of removed items comprising the item;
presenting, by the computing system, the log of removed items on a display of the smart crash cart; and
notifying, by the computing system, the central server of the log of removed items.

11. The computer-implemented method of claim 10, further comprising:
determining, by the computing system based on the log of removed items, an inventory of a category of the one or more items being depleted; and
notifying, via a second push notification by the computing system, the central server of the category being depleted.

12. The computer-implemented method of claim 10, further comprising:
sensing, via one or more second radio signals disposed on a waste bin of the smart crash cart, placement of the item into the waste bin;
confirming, by the computing system via the information of the item, the removal of the item from the smart crash cart;
updating, by the computing system, the log of removed items to indicate placement of the item into the waste bin.

13. The computer-implemented method of claim 8, wherein the one or more chips are no more than about 10 cm².

14. The computer-implemented method of claim 8, wherein the one or more items comprise at least one of medications, medical instruments, airway equipment, and defibrillator equipment.

15. A non-transitory computer readable medium storing instructions, that when executed by one or more processors, cause the one or more processors to implement the instructions, the instructions comprising:
providing one or more chips on one or more items in a smart crash cart, each of the one or more chips containing information relating to an item of the one or more items, the information including an expiration date of the item;
sensing, via one or more radio signal sensors, radio signals from the one or more chips;
determining a difference between a current date and the expiration date of the item;
comparing the difference with a threshold difference;
determining the difference is at the threshold difference;
notifying, via a push notification, a central server of the expiration date of the item, the central server configured to communicate with the smart crash cart;
sensing, via the one or more radio signal sensors, removal of the item from the smart crash cart;
sensing, via the one or more radio signal sensors, replacing of the item in the smart crash cart;
generating an alert of the replaced item; and
transmitting the alert to the central server.

16. The non-transitory computer readable medium of claim 15, the instructions further comprising:
tracking a location of the one or more items via the one or more chips.

17. The non-transitory computer readable medium of claim 15, the instructions further comprising:
generating a log of removed items comprising the item;
presenting the log of removed items on a display of the smart crash cart; and
notifying the central server of the log of removed items.

18. The non-transitory computer readable medium of claim 17, the instructions further comprising:
determining, based on the log of removed items, an inventory of a category of the one or more items being depleted; and
notifying, via a second push notification, the central server of the category being depleted.

19. The non-transitory computer readable medium of claim 17, the instructions further comprising:
sensing, via one or more second radio signals disposed on a waste bin of the smart crash cart, placement of the item into the waste bin;
confirming, via the information of the item, the removal of the item from the smart crash cart;
updating the log of removed items to indicate placement of the item into the waste bin.

20. The non-transitory computer readable medium of claim 15, wherein the one or more items comprise at least one of medications, medical instruments, airway equipment, and defibrillator equipment.
